(19) United States
(12) Patent Application Publication
(10) Pub. No.: US 2006/0148877 A1
(43) Pub. Date: Jul. 6, 2006

(54) Pharmaceutical formulations of celcoxib

(75) Inventors: Steven Bernstein, Philadelphia, PA (US); Hongming Chen, Acton, MA (US); Colin R. Gardner, Concord, MA (US); Meegan LeMott, East Lyme, CT (US); Szu-Wen Wang, Irvine, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: TransForm Pharmaceuticals, Inc., Lexington, MA (US)

(21) Appl. No.: 10/534,612

(22) PCT Filed: Nov. 25, 2003
(86) PCT No.: PCT/US03/37479

§ 371 Date: Nov. 18, 2005

Related U.S. Application Data
(60) Provisional application No. 60/429,515, filed on Nov. 26, 2002.

(51) Int. Cl. A61K 31/415; A61K 36/47

(52) U.S. Cl. 514/406; 424/731

(57) ABSTRACT

Liquid formulations of celecoxib have been found to provide faster pain relief than conventional solid formulations of celecoxib. The present invention provides combinations of excipients in which celecoxib is highly soluble for formulation as pharmaceutical compositions.

### BACKGROUND OF THE INVENTION

Celecoxib (4-[5-(4-methylphenyl)-3-(trifluoromethyl)-1H-pyrazol-1-yl]benzenesulfonamide) is a substituted pyrazolylbenzenesulfonamide represented by the structure: 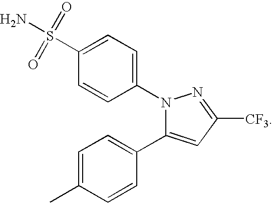
Celecoxib belongs to the general class of non-steroidal anti-inflammatory drugs (NSAIDs). Unlike traditional NSAIDs, celecoxib is a selective inhibitor of cyclooxygenase II (COX-2) that causes fewer side effects when administered to a subject. The synthesis and use of celecoxib are further described in U.S. Pat. Nos. 5,466,823, 5,510,496, 5,563,165, 5,753,688, 5,760,068, 5,972,986, and 6,156,781, the contents of which are incorporated by reference in their entireties.

In its commercially available form as CELEBREX®, celecoxib is a neutral molecule that is essentially insoluble in water. Celecoxib typically exists as needle-like crystals, which tend to aggregate into a mass. This can present significant problems in preparing pharmaceutical formulations of celecoxib, particularly oral formulations.

Liquid formulations of celecoxib have been shown to have faster absorption in animal studies when compared to solid formulations. Liquid formulations have also been shown to achieve higher blood plasma concentrations when compared to solid formulations. Both of these effects combined enable liquid formulations of celecoxib to give faster onset of pain relief.

Thus, it is desirable to find a combination of pharmaceutically acceptable excipients which have a celecoxib solubility of at least 200 mg/mL. Ideally, a liquid formulation should be administered in a small, reproducible volume (e.g., a softgel capsule) to insure proper dosing and maximize patient compliance. This solubility level would provide a dosage equivalent of reasonable size to that of the strongest currently available dosage form, a 200 mg capsule.

### SUMMARY OF THE INVENTION

It has now been been found that celecoxib is particularly soluble (at least 200 mg/mL) in several combinations of pharmaceutically acceptable excipients. The present invention discloses a number of such combinations of pharmaceutically acceptable excipients.

In one embodiment, the present invention is a pharmaceutical composition comprising celecoxib; a first excipient which is a propylene glycol fatty acid monoester; a second excipient selected from the group consisting of a polyethylene glycol, a polyoxyl castor oil, and a polysorbate; and a third excipient selected from the group consisting of a propylene glycol fatty acid monoester, a polysorbate, a sorbitan fatty acid monoester, a poloxamer, a polyethylene glycol, a polyethylene glycol fatty acid monoester, and a polyoxyl castor oil.

In another embodiment, the present invention is a pharmaceutical composition comprising celecoxib; a first excipient which is a poloxamer; a second excipient selected from the group consisting of a propylene glycol fatty acid monoester, a polyoxamer, and a plant oil; and a third excipient selected from the group consisting of a propylene glycol fatty acid monoester, a poloxamer, a polysorbate, and a sorbitan fatty acid monoester.

In yet another embodiment, the present invention is a pharmaceutical composition comprising celecoxib; a first excipient which is a polyethylene glycol; a second excipient selected from the group consisting of a polyethylene glycol, a polyoxyl castor oil, and a sorbitan fatty acid monoester; and a third excipient selected from the group consisting of a polyethylene glycol, a sorbitan fatty acid monoester, and a propylene glycol fatty acid monoester.

In one aspect, the present invention is a pharmaceutical composition comprising celecoxib; a first excipient which is a sorbitan fatty acid monoester; a second excipient selected from the group consisting of a polyethylene glycol and a polysorbate; and a third excipient selected from the group consisting of a propylene glycol fatty acid monoester, a polyoxamer, a polyethylene glycol, a polyoxyl castor oil, a polysorbate, a plant oil, and a sorbitan fatty acid monoester.

In another aspect, the present invention is a pharmaceutical composition comprising celecoxib; a first excipient which is a polyoxyl castor oil; a second excipient selected from the group consisting of a propylene glycol fatty acid monoester, a poloxamer, a polyethylene glycol, a polyoxyl castor oil, a polysorbate, and a sorbitan fatty acid monoester; and a third excipient selected from the group consisting of a propylene glycol fatty acid monoester, a polyoxamer, a polyethylene glycol, a polyoxyl castor oil, a polysorbate, a plant oil, and a sorbitan fatty acid monoester.

In yet another aspect, the present invention is a pharmaceutical composition comprising celecoxib; a first excipient which is a polysorbate; a second excipient selected from the group consisting of a propylene glycol fatty acid monoester, a poloxamer, a polyethylene glycol, a polyoxyl castor oil, a polysorbate, and a sorbitan fatty acid monoester; and a third excipient selected from the group consisting of a propylene glycol fatty acid monoester, a polyethylene glycol, a polyoxyl castor oil, a polysorbate, a plant oil, a sorbitan fatty acid monoester, and a poloxamer.

The present invention provides a pharmaceutical composition comprising celecoxib; a first excipient which is polyoxyl 35 castor oil; and a second excipient which is selected from the group consisting of a propylene glycol fatty acid monoester, a polyethylene glycol, a polyoxyl castor oil, a polysorbate, a sorbitan fatty acid monoester, mono- and di-glycerides from plant oil, a monoglyceride of a hydroxylated fatty acid, a tricarboxylic acid, tocopherol, and a polyethylene glycol fatty acid ester.

The present invention also provides a pharmaceutical composition comprising celecoxib; a first excipient which is poloxamer 331; and a second excipient selected from the group consisting of a plant oil, mono- and di-glycerides from plant oil, a tricarboxylic acid, and a polyethylene glycol fatty acid ester.

The present invention further provides a pharmaceutical composition comprising celecoxib; a first excipient which is polyethylene glycol 400; and a second excipient selected from the group consisting of a polysorbate, a sorbitan fatty acid monoester, mono- and di-glycerides from plant oil, a monoglyceride of a hydroxylated fatty acid, a tricarboxylic acid, a trialkanolamine, and a polyethylene glycol fatty acid ester.

In one embodiment, the present invention is a pharmaceutical composition comprising celecoxib; a first excipient which is polyethylene glycol 60 almond glycerides; and a second excipient selected from the group consisting of a propylene glycol fatty acid monoester, a polyethylene glycol, a polyoxyl castor oil, a polysorbate, a sorbitan fatty acid monoester, mono- and di-glycerides from plant oil, a monoglyceride of a hydroxylated fatty acid, a tricarboxylic acid, tocopherol, and a polyethylene glycol fatty acid ester.

In another embodiment, the present invention is a pharmaceutical composition comprising celecoxib; a first excipient which is polyethylene glycol 6 isostearate; and a second excipient selected from the group consisting of a propylene glycol fatty acid monoester, a poloxamer, a polyoxyl castor oil, a polysorbate, a plant oil, mono- and di-glycerides from plant oil, a monoglyceride of a hydroxylated fatty acid, a tricarboxylic acid, tocopherol, and a polyethylene glycol fatty acid ester.

In yet another embodiment, the present invention is a pharmaceutical composition comprising celecoxib; a first excipient which is C₈ and C₁₀ mono- and di-glycerides from coconut oil; and a second excipient selected from the group consisting of a polyethylene glycol and a polyethylene glycol fatty acid ester.

In one aspect, the present invention is a pharmaceutical composition comprising celecoxib; a first excipient which is triacetin; and a second excipient selected from the group consisting of a polyethylene glycol, a polyoxyl castor oil, and a polyethylene glycol fatty acid ester.

In another aspect, the present invention is a pharmaceutical composition comprising celecoxib; a first excipient which is triethanolamine; and a second excipient selected from the group consisting of a polyethylene glycol and mono- and di-glycerides from plant oil.

In yet another aspect, the present invention is a pharmaceutical composition comprising celecoxib; tocopherol; and a polyethylene glycol.

The present invention provides a pharmaceutical composition comprising celecoxib; glyceryl ricinoleate; and a polysorbate.

The present invention also provides a pharmaceutical composition comprising celecoxib; a first excipient which is C₈ and C₁₀ mono- and di-glycerides from coconut oil; a second excipient which is triacetin; and a third excipient which is a mixture containing about 90% by weight monoolein and about 10% by weight propylene glycol.

The present invention further provides a pharmaceutical composition comprising celecoxib; a first excipient which is triacetin; a second excipient selected from the group consisting of acetylated monoglycerides, a mixture containing about 90% by weight monoolein and about 10% by weight propylene glycol, and triacetin; and a third excipient selected from the group consisting of acetylated monoglycerides, glyceryl trilaurate, triacetin, and triethanolamine.

The present invention also includes a method of preparing a pharmaceutical composition disclosed herein, comprising the step of dissolving celecoxib in a mixture of excipients disclosed herein in an appropriate ratio. Appropriate ratios are also disclosed herein.

In another embodiment, the present invention includes a method of treating a subject in need of celecoxib, comprising the step of administering to the subject one of the pharmaceutical compositions disclosed herein. A subject in need of celecoxib typically is suffering from one of the ailments listed below. Subjects particularly in need of celecoxib include those suffering from acute pain (e.g., pain that needs to be relieved with 30 minutes), rheumatoid arthritis, osteoarthritis and primary dysmenorrhea. Pharmaceutical compositions of the invention are typically administered orally with the active agent in a liquid form, such as in a capsule or syrup.

Advantages of the present invention include the ability to solubilize high concentrations of celecoxib, which allows a large dose of celecoxib to be administered in a manageable volume. In addition, liquid formulations of celecoxib have been shown to provide pain relief more rapidly.

### DETAILED DESCRIPTION OF THE INVENTION

The present invention provides a number of formulations of pharmaceutically acceptable excipients in which celecoxib is highly soluble, typically greater than 200 mg/mL. Because of the structural similarities between molecules, the combinations of excipients disclosed herein are also intended to provide formulations for solubilizing other COX-2 selective inhibitors, which can be used to treat subjects suffering from the conditions disclosed below. COX-2 selective inhibitors are described, for example, in U.S. Pat. Nos. 5,344,991, 5,380,738, 5,393,790, 5,401,765, 5,418,254, 5,420,343, 5,434,178, 5,436,265, 5,466,823, 5,474,995, 5,475,018, 5,486,534, 5,510,368, 5,521,213, 5,536,752, 5,543,297, 5,547,975, 5,550,142, 5,552,422, 5,585,504, 5,593,992, 5,596,008, 5,604,253, 5,604,260, 5,616,458, 5,616,601, 5,620,999, 5,633,272, 5,639,780, 5,643,933, 5,658,903, 5,668,161, 5,670,510, 5,677,318, 5,681,842, 5,686,460, 5,686,470, 5,696,143, 5,710,140, 5,716,955, 5,723,485, 5,739,166, 5,741,798, 5,756,499, 5,756,529, 5,776,967, 5,783,597, 5,789,413, 5,807,873, 5,817,700, 5,830,911, 5,849,943, 5,859,036, 5,861,419, 5,866,596, 5,869,524, 5,869,660, 5,883,267, 5,892,053, 5,922,742, 5,929,076, 5,932,598, 5,935,990, 5,945,539, 5,958,978, 5,968,958, 5,972,950, 5,973,191, 5,981,576, 6,002,014, 6,004,960, 6,005,000, 6,020,343, 6,020,347, 6,034,256, 6,040,319, 6,040,450, 6,046,208, 6,046,217, 6,057,319, 6,063,804, 6,063,807, 6,071,954, 6,077,868, 6,083,969, 6,096,753 and 6,133,292, as well as European Patent Application Nos. 0799823, 0846689, 0863134 and 0985666, and PCT Publication Nos. WO 94/15932, WO 96/19469, WO 96/26921, WO 96/31509, WO 96/36623, WO 96/38418, WO 97/03953, WO 97/10840, WO 97/13755, WO 97/13767, WO 97/25048, WO 97/30030, WO 97/34882, WO 97/46524, WO 98/04527, WO 98/06708, WO 98/07425, WO 98/17292, WO 98/21195, WO 98/22457, WO 98/32732, WO 98/41516, WO 98/43966, WO 98/45294, WO 98/47871, WO 99/01130, WO 99/01131, WO 99/01452, WO 99/01455, WO 99/01455, WO 99/10331, WO 99/10332, WO 99/11605, WO 99/12930, WO 99/14195, WO 99/14205, WO 99/15505, WO 99/23087, WO 99/24404, WO 99/25695, WO 99/35130, WO 99/61016, WO 99/61436, WO 99/62884, WO 99/64415, WO 00/01380, WO 00/08024, WO 00/10993, WO 00/13684, WO 00/18741, WO 00/18753, WO 00/23426, WO 00/24719, WO 00/26216, WO 00/31072, WO 00/40087 and WO 00/56348, the contents of each of which are incorporated herein by reference. In particular, the present invention is intended to include selective COX-2 inhibitors valdecoxib and rofecoxib.

Fatty acids, as defined herein, include saturated and unsaturated fatty acid (e.g., mono-, di-, and multi-unsaturated fatty acids). Saturated fatty acids include lauric acid, myristic acid, palmitic acid, stearic acid, arachidic acid, behenic acid and lignoceric acid. Unsaturated fatty acids include palmitoleic acid, oleic acid, linoleic acid, alpha-linolenic acid, gamma-linolenic acid, arachidonic acid, elaidic acid and nervonic acid. The fatty acids listed here can optionally be hydroxylated to obtain, for example, ricinoleic acid.

First excipients of the present invention, when there are three excipients into which celecoxib or another COX-2 inhibitor is dissolved, typically comprise about 60% to about 70% by weight, or preferably about 63% to about 67% by weight, of the three excipients. The second excipient, when there are three excipients into which celecoxib or another COX-2 inhibitor is dissolved, typically comprises about 20% to about 30% by weight, or preferably about 24% to about 28% by weight, of the three excipients. The third excipient, when there are three excipients into which celecoxib or another COX-2 inhibitor is dissolved, typically comprises about 5% to about 15% by weight, or preferably about 8% to about 12% by weight, of the three excipients.

First excipients of the present invention, when there are two excipients into which celecoxib or another COX-2 inhibitor is dissolved, typically comprise about 70% to about 80% by weight, or preferably about 73% to about 77% by weight, of the two excipients. The second excipient, when there are two excipients into which celecoxib or another COX-2 inhibitor is dissolved, typically comprises about 20% to about 30% by weight, or preferably about 23% to about 27% by weight, of the two excipients.

Plant oils include sesame oil, peanut oil, coconut oil, corn oil, olive oil, palm oil, safflower oil, soybean oil and sunflower oil.

The uptake of a drug by a subject can be assessed in terms of maximum blood serum concentration and time to reach maximum blood serum concentration. Pharmaceutical compositions with a more rapid onset to therapeutic effect typically reach a higher maximum blood serum concentration (Cₘₐₓ) a shorter time after oral administration (Tₘₐₓ). Preferably, pharmaceutical compositions of the present invention have a shorter Tₘₐₓ than presently-marketed celecoxib. Even more preferably, the therapeutic effects of pharmaceutical compositions of the present invention begin to occur within about 30 minutes, within about 25 minutes, within about 20 minutes, within about 15 minutes, within about 10 minutes, or within about 5 minutes of administration (e.g., oral administration). Ailments treatable with pharmaceutical compositions of the present invention are discussed below. Treatment of pain is a preferred embodiment of the present invention.

Excipients employed in pharmaceutical compositions of the present invention include, but are not limited to: acetylated monoglycerides, monoolein:propylene glycol (90:10), mono-/diglyceride from coconut oil (C8/C10), propylene glycol monocaprylate, caprylic/capric triglyceride, C8/C10 diesters of propylene glycol of coconut oil, castor oil, coconut oil, corn oil, cottonseed oil, PEG 60 almond glycerides, diacetylated monoglycerides, ethylene glycol, gelucire 33/01, glycerin, glyceryl linoleate, glyceryl oleate, glyceryl ricinoleate, hydrogenated coconut oil, oleoyl macrogol-6 glycerides;apricot kernel oil PEG-6 ester, linoleoyl macrogol-6 glycerides;corn oil PEG-6 ester, PEG-8 caprylic/capric glyceride;caprylocaproyl macrogol-8 glycerides, propylene glycol monolaurate, lecithin (high HLB), lecithin (low HLB), linoleic acid, mineral oil, myristyl alcohol, oleic acid, PEG-6 isostearate, olive oil, palm oil (palm butter), peanut oil, polyglycerol-3-diisostearate, polyglyceryl-6-dioleate, Ethosperse G-26 (Lonza), poloxamer 331, polyethylene glycol 1000 (PEG-20), polyethylene glycol 200, polyethylene glycol 300, polyethylene glycol 400, polyethylene glycol 600, polyoxyl 20 stearate, polyoxyl 30 castor oil, polyoxyl 35 castor oil, polyoxyl 40 castor oil, polyoxyl 40 hydrogenated castor oil, polyoxyl 40 stearate, polypropylene glycol (MW 725), polypropylene glycol (MW 2000), polysorbate 20, polysorbate 40, polysorbate 80, polysorbate 60, propylene glycol, safflower oil, sesame oil, sorbitan monolaurate, sorbitan monooleate, sorbitan trioleate, soybean oil, sunflower seed oil, polyoxyethylene glycerol trioleate, tocopherol, triacetin, triethanolamine, trilaurin (glyceryl trilaurate), vegetable oil (partially hydrogenated and hydrogenated), vitamin E TPGS, benzyl alcohol, benzyl benzoate, ethylene glycol monoethyl ether, isopropanolamine, and diethylene glycol monoethyl ether.

Excipients are combined with celecoxib to provide formulations of the invention. Typical excipients are liquids or semi-solids at room temperature, although they may be highly viscous. Semi-solids are defined as materials with a melting point between room temperature and 40 degrees C. As used herein and unless otherwise specified, the term “liquid” includes those materials defined as semi-solids. A liquid excipient, according to the present invention, includes excipients with a melting point below 40 degrees C. Moreover, solid excipients can be dissolved in liquid excipients.

Excipients may be novel, but commercially available excipients that are generally recognized as safe (GRAS) are preferably used. Formulations can comprise one or more excipients. For example, a formulation can comprise any one, any two, any three, any four, any five, or more excipients listed herein (e.g., above), in addition to celecoxib. A binary formulation comprises two excipients and celecoxib, a ternary formulation comprises three excipients and celecoxib, and so on. Each combination is included as an individual species of the present invention. Each species may also be specifically excluded from the present invention.

Excipient mixtures, according to the present invention, can comprise many ratios of two or more excipients. For example, a binary excipient mixture can comprise about 1, 2, 3, 4, 5, 7, 10, 15, 20, 25, 30, 35, 40 ,45, 50, 55, 60, 65, 70, 75, 80, 85, 90, 95, 98, or about 99 percent by weight or volume of excipient A and comprise about 1, 2, 3, 4, 5, 7, 10, 15, 20, 25, 30, 35, 40 ,45, 50, 55, 60, 65, 70, 75, 80, 85, 90, 95, 98, or about 99 percent by weight or volume of excipient B. Some specific ratios of binary excipient mixtures include, but are not limited to, 1:1, 2:1, 1.5:1, 3:1, etc. Similarly, a ternary mixture can comprise about 1, 2, 3, 4, 5, 7, 10, 15, 20, 25, 30, 35, 40 ,45, 50, 55, 60, 65, 70, 75, 80, 85, 90, 95, 98, or about 99 percent by weight or volume of excipients A, B, and C. Some specific ratios of ternary excipient mixtures include, but are not limited to, 1:1:1, 2:1:1, 2:1:0.5, 1:1:0.5, etc. Higher order mixtures and ratios of excipients (e.g., quarternary) are also included in the present invention.

As detailed in the examples, many combinations of two, three or more excipients have been shown to solubilize celecoxib in an unexpectedly high amount. For example, many binary and ternary excipient mixtures have been shown to dissolve celecoxib at surprisingly high concentrations of 100 mg/mL, or at 200 mg/mL. These mixtures of celecoxib dissolved in liquid excipients can be administered in small, reproducible volumes (e.g., a softgel capsule) to a subject in need of pain relief. Moreover, the high concentration of celecoxib allowed by these particular excipient mixtures enables ease of dosing and can improve subject compliance.

According to the present invention, the excipients described herein can be used to prepare liquid formulations of celecoxib at celecoxib concentrations greater than 50 mg/mL, 75 mg/mL, 100 mg/mL, 125 mg/mL, 150, mg/mL, 175 mg/mL, or 200 mg/mL, both at room temperature and at physiological temperature.

Liquid formulations of celecoxib can be administered by controlled- or delayed-release means. Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. (Kim, Cherng-ju, Controlled Release Dosage Form Design, 2 (Technomic Publishing, Lancaster, Pa.: 2000)).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under dosing a drug (i.e., going below the minimum therapeutic levels) as well as exceeding the toxicity level for the drug.

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the liquid formulations and compositions of the invention. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions.

One embodiment of the invention encompasses a unit dosage form which comprises a celecoxib formulation of the present invention, and one or more pharmaceutically acceptable excipients or diluents, wherein the pharmaceutical composition or dosage form is formulated for controlled-release. Specific dosage forms utilize an osmotic drug delivery system.

A particular and well-known osmotic drug delivery system is referred to as OROS® (Alza Corporation, Mountain View, Calif. USA). This technology can readily be adapted for the delivery of compounds and compositions of the invention. Various aspects of the technology are disclosed in U.S. Pat. Nos. 6,375,978 B1; 6,368,626 B1; 6,342,249 B1; 6,333,050 B2; 6,287,295 B1; 6,283,953 B1; 6,270,787 B1; 6,245,357 B1; and 6,132,420; each of which is incorporated herein by reference. Specific adaptations of OROS® that can be used to administer compounds and compositions of the invention include, but are not limited to, the OROS® Push-Pull™, Delayed Push-Pull™, Multi-Layer Push-Pull™, and Push-Stick™ Systems, all of which are well known. See, e.g., http://www.alza.com. Additional OROS® systems that can be used for the controlled oral delivery of compounds and compositions of the invention include OROS®-CT and L-OROS®. Id.; see also, Delivery Times, vol. II, issue II (Alza Corporation).

Conventional OROS® oral dosage forms are made by compressing a drug powder into a hard tablet, coating the tablet with cellulose derivatives to form a semi-permeable membrane, and then drilling an orifice in the coating (e.g., with a laser). (Kim, Cherng-ju, Controlled Release Dosage Form Design, 231-238 (Technomic Publishing, Lancaster, Pa. 2000)). Liquid formulations of the present invention can be combined with a pharmaceutically acceptable carrier (e.g. lactose) to form a paste or a solid suitable for such controlled release oral dosage forms. The advantage of such dosage forms is that the delivery rate of the drug is not influenced by physiological or experimental conditions. Even a drug with a pH-dependent solubility can be delivered at a constant rate regardless of the pH of the delivery medium. But because these advantages are provided by a build-up of osmotic pressure within the dosage form after administration, conventional OROS® drug delivery systems cannot be used to effectively deliver drugs with low water solubility.

A specific dosage form of the invention comprises: a wall defining a cavity, the wall having an exit orifice formed or formable therein and at least a portion of the wall being semipermeable; an expandable layer located within the cavity remote from the exit orifice and in fluid communication with the semipermeable portion of the wall; a dry or substantially dry state drug layer located within the cavity adjacent to the exit orifice and in direct or indirect contacting relationship with the expandable layer; and a flow-promoting layer interposed between the inner surface of the wall and at least the external surface of the drug layer located within the cavity, wherein the drug layer comprises a liquid formulation of celecoxib. See U.S. Pat. No. 6,368,626, the entirety of which is incorporated herein by reference.

Another specific dosage form of the invention comprises: a wall defining a cavity, the wall having an exit orifice formed or formable therein and at least a portion of the wall being semipermeable; an expandable layer located within the cavity remote from the exit orifice and in fluid communication with the semipermeable portion of the wall; a drug layer located within the cavity adjacent the exit orifice and in direct or indirect contacting relationship with the expandable layer; the drug layer comprising a liquid, active agent formulation absorbed in porous particles, the porous particles being adapted to resist compaction forces sufficient to form a compacted drug layer without significant exudation of the liquid, active agent formulation, the dosage form optionally having a placebo layer between the exit orifice and the drug layer, wherein the active agent formulation comprises a liquid formulation of celecoxib. See U.S. Pat. No. 6,342,249, the entirety of which is incorporated herein by reference. Celecoxib dosage forms of the invention preferably comprise celecoxib in a daily dosage amount of about 10 mg to about 1000 mg, more preferably about 25 mg to about 400 mg, and most preferably about 50 mg to about 200 mg.

Pharmaceutical compositions of the invention comprise one or more orally deliverable dose units. Each dose unit comprises celecoxib in a therapeutically effective amount that is preferably about 10 mg to about 1000 mg. The term “dose unit” herein means a portion of a pharmaceutical composition that contains an amount of a therapeutic or prophylactic agent, in the present case celecoxib, suitable for a single oral administration to provide a therapeutic effect. Typically one dose unit, or a small plurality (up to about 4) of dose units, in a single administration provides a dose comprising a sufficient amount of the agent to result in the desired effect. Administration of such doses can be repeated as required, typically at a dosage frequency of 1 to about 4 times per day.

It will be understood that a therapeutically effective amount of celecoxib for a subject is dependent inter alia on the body weight of the subject. A “subject” to which a pharmaceutical composition of the present invention can be administered includes a human subject of either sex and of any age, and also includes any nonhuman animal, particularly a warm-blooded animal, more particularly a domestic or companion animal, illustratively a cat, dog or horse. When the subject is a child or a small animal (e.g., a dog), for example, an amount of celecoxib relatively low in the preferred range of about 10 mg to about 1000 mg is likely to provide blood serum concentrations consistent with therapeutic effectiveness. Where the subject is an adult human or a large animal (e.g., a horse), achievement of such blood serum concentrations of celecoxib is likely to require dose units containing a relatively greater amount of celecoxib.

Typical dose units in a pharmaceutical composition of the invention contain about 10, 20, 25, 37.5, 50, 75, 100, 125, 150, 175, 200, 250, 300, 350 or 400 mg of celecoxib. For an adult human, a therapeutically effective amount of celecoxib per dose unit in a composition of the present invention is typically about 50 mg to about 400 mg. Especially preferred amounts of celecoxib per dose unit are about 100 mg to about 200 mg, for example about 100 mg or about 200 mg. Other doses that are not in current use for CELEBREX® may become preferred, if the bioavailability is changed with a novel formulation. For instance, 300 mg may become a preferred dose for certain indications.

A dose unit containing a particular amount of celecoxib can be selected to accommodate any desired frequency of administration used to achieve a desired daily dosage. The daily dosage and frequency of administration, and therefore the selection of appropriate dose unit, depends on a variety of factors, including the age, weight, sex and medical condition of the subject, and the nature and severity of the condition or disorder, and thus may vary widely.

For pain management, pharmaceutical compositions of the present invention can be used to provide a daily dosage of celecoxib of about 50 mg to about 1000 mg, preferably about 100 mg to about 600 mg, more preferably about 150 mg to about 500 mg, and still more preferably about 175 mg to about 400 mg, for example about 200 mg. A daily dose of celecoxib of about 0.7 to about 13 mg/kg body weight, preferably about 1.3 to about 8 mg/kg body weight, more preferably about 2 to about 6.7 mg/kg body weight, and still more preferably about 2.3 to about 5.3 mg/kg body weight, for example about 2.7 mg/kg body weight, is generally appropriate when administered in a pharmaceutical composition of the invention. The daily dose can be administered in one to about four doses per day. Administration at a rate of one 50 mg dose unit four times a day, one 100 mg dose unit or two 50 mg dose units twice a day or one 200 mg dose unit, two 100 mg dose units or four 50 mg dose units once a day is preferred.

The term “oral administration” herein includes any form of delivery of a therapeutic agent or a composition thereof to a subject wherein the agent or composition is placed in the mouth of the subject, whether or not the agent or composition is immediately swallowed. Thus, “oral administration” includes buccal and sublingual as well as esophageal administration. Absorption of the agent can occur in any part or parts of the gastrointestinal tract including the mouth, esophagus, stomach, duodenum, ileum and colon. The term “orally deliverable” herein means suitable for oral administration.

Pharmaceutical compositions of the invention are useful in treatment and prevention of a very wide range of disorders mediated by COX-2, including but not restricted to disorders characterized by inflammation, pain and/or fever. Such pharmaceutical compositions are especially useful as anti-inflammatory agents, such as in treatment of arthritis, with the additional benefit of having significantly less harmful side effects than compositions of conventional non-steroidal anti-inflammatory drugs (NSAIDs) that lack selectivity for COX-2 over COX-1. In particular, pharmaceutical compositions of the invention have reduced potential for gastrointestinal toxicity and gastrointestinal irritation including upper gastrointestinal ulceration and bleeding, reduced potential for renal side effects such as reduction in renal function leading to fluid retention and exacerbation of hypertension, reduced effect on bleeding times including inhibition of platelet function, and possibly a lessened ability to induce asthma attacks in aspirin-sensitive asthmatic subjects, by comparison with compositions of conventional NSAIDs. Thus compositions of the invention are particularly useful as an alternative to conventional NSAIDs where such NSAIDs are contraindicated, for example in subjects with peptic ulcers, gastritis, regional enteritis, ulcerative colitis, diverticulitis or with a recurrent history of gastrointestinal lesions; gastrointestinal bleeding, coagulation disorders including anaemia such as hypoprothrombinemia, hemophilia or other bleeding problems; kidney disease; or in subjects prior to surgery or subjects taking anticoagulants.

Contemplated pharmaceutical compositions are useful to treat a variety of arthritic disorders, including but not limited to rheumatoid arthritis, spondyloarthropathies, gouty arthritis, osteoarthritis, systemic lupus erythematosus and juvenile arthritis.

Such pharmaceutical compositions are useful in treatment of asthma, bronchitis, menstrual cramps, preterm labor, tendonitis, bursitis, allergic neuritis, cytomegalovirus infectivity, apoptosis including HIV-induced apoptosis, lumbago, liver disease including hepatitis, skin-related conditions such as psoriasis, eczema, acne, burns, dermatitis and ultraviolet radiation damage including sunburn, and post-operative inflammation including that following ophthalmic surgery such as cataract surgery or refractive surgery.

Pharmaceutical compositions of the present invention are useful to treat gastrointestinal conditions such as, but not limited to, inflammatory bowel disease, Crohn's disease, gastritis, irritable bowel syndrome and ulcerative colitis.

Such pharmaceutical compositions are useful in treating inflammation in such diseases as migraine headaches, periarteritis nodosa, thyroiditis, aplastic anemia, Hodgkin's disease, sclerodoma, rheumatic fever, type I diabetes, neuromuscular junction disease including myasthenia gravis, white matter disease including multiple sclerosis, sarcoidosis, nephrotic syndrome, Behcet's syndrome, polymyositis, gingivitis, nephritis, hypersensitivity, swelling occurring after injury including brain edema, myocardial ischemia, and the like.

In addition, these pharmaceutical compositions are useful in treatment of ophthalmic diseases, such as retinitis, conjunctivitis, retinopathies, uveitis, ocular photophobia, and of acute injury to the eye tissue.

Also, such pharmaceutical compositions are useful in treatment of pulmonary inflammation, such as that associated with viral infections and cystic fibrosis, and in bone resorption such as that associated with osteoporosis.

The pharmaceutical compositions are useful for treatment of certain central nervous system disorders, such as cortical dementias including Alzheimer's disease, neurodegeneration, and central nervous system damage resulting from stroke, ischemia and trauma. The term “treatment” in the present context includes partial or total inhibition of dementias, including Alzheimer's disease, vascular dementia, multi-infarct dementia, pre-senile dementia, alcoholic dementia and senile dementia.

Such pharmaceutical compositions are useful in treatment of allergic rhinitis, respiratory distress syndrome, endotoxin shock syndrome and liver disease.

Further, pharmaceutical compositions of the present invention are useful in treatment of pain, including but not limited to postoperative pain, dental pain, muscular pain, and pain resulting from cancer. For example, such compositions are useful for relief of pain, fever and inflammation in a variety of conditions including rheumatic fever, influenza and other viral infections including common cold, low back and neck pain, dysmenorrhea, headache, toothache, sprains and strains, myositis, neuralgia, synovitis, arthritis, including rheumatoid arthritis, degenerative joint diseases (osteoarthritis), gout and ankylosing spondylitis, bursitis, bums, and trauma following surgical and dental procedures.

The present invention is further directed to a therapeutic method of treating a condition or disorder where treatment with a COX-2 inhibitory drug is indicated, the method comprising oral administration of a pharmaceutical composition of the invention to a subject in need thereof. The dosage regimen to prevent, give relief from, or ameliorate the condition or disorder preferably corresponds to once-a-day or twice-a-day treatment, but can be modified in accordance with a variety of factors. These include the type, age, weight, sex, diet and medical condition of the subject and the nature and severity of the disorder. Thus, the dosage regimen actually employed can vary widely and can therefore deviate from the preferred dosage regimens set forth above. The present pharmaceutical compositions can be used in combination with other therapies or therapeutic agents, including but not limited to, therapies with opioids and other analgesics, including narcotic analgesics, Mu receptor antagonists, Kappa receptor antagonists, non-narcotic (i.e. non-addictive) analgesics, monoamine uptake inhibitors, adenosine regulating agents, cannabinoid derivatives, GABA active agents, norexin neuropeptide modulators, Substance P antagonists, neurokinin-1 receptor antagonists and sodium channel blockers, among others. Preferred combination therapies comprise use of a composition of the invention with one or more compounds selected from aceclofenac, acemetacin, e-acetamidocaproic acid, acetaminophen, acetaminosalol, acetanilide, acetylsalicylic acid (aspirin), S-adenosylmethionine, alclofenac, alfentanil, allylprodine, alminoprofen, aloxiprin, alphaprodine, aluminum bis(acetylsalicylate), amfenac, aminochlorthenoxazin, 3-amino-4-hydroxybutyric acid, 2-amino-4-picoline, aminopropylon, aminopyrine, amixetrine, ammonium salicylate, ampiroxicam, amtolmetin guacil, anileridine, antipyrine, antipyrine salicylate, antrafenine, apazone, bendazac, benorylate, benoxaprofen, benzpiperylon, benzydamine, benzylmorphine, bermoprofen, bezitramide, alpha-bisabolol, bromfenac, p-bromoacetanilide, 5-bromosalicylic acid acetate, bromosaligenin, bucetin, bucloxic acid, bucolome, bufexamac, bumadizon, buprenorphine, butacetin, butibufen, butophanol, calcium acetylsalicylate, carbamazepine, carbiphene, carprofen, carsalam, chlorobutanol, chlorthenoxazin, choline salicylate, cinchophen, cinmetacin, ciramadol, clidanac, clometacin, clonitazene, clonixin, clopirac, clove, codeine, codeine methyl bromide, codeine phosphate, codeine sulfate, cropropamide, crotethamide, desomorphine, dexoxadrol, dextromoramide, dezocine, diampromide, diclofenac sodium, difenamizole, difenpiramide, diflunisal, dihydrocodeine, dihydrocodeinone enol acetate, dihydromorphine, dihydroxyaluminum acetylsalicylate, dimenoxadol, dimepheptanol, dimethylthiambutene, dioxaphetyl butyrate, dipipanone, diprocetyl, dipyrone, ditazol, droxicam, emorfazone, enfenamic acid, epirizole, eptazocine, etersalate, ethenzamide, ethoheptazine, ethoxazene, ethylmethylthiambutene, ethylmorphine, etodolac, etofenamate, etonitazene, eugenol, felbinac, fenbufen, fenclozic acid, fendosal, fenoprofen, fentanyl, fentiazac, fepradinol, feprazone, floctafenine, flufenamic acid, flunoxaprofen, fluoresone, flupirtine, fluproquazone, flurbiprofen, fosfosal, gentisic acid, glafenine, glucametacin, glycol salicylate, guaiazulene, hydrocodone, hydromorphone, hydroxypethidine, ibufenac, ibuprofen, ibuproxam, imidazole salicylate, indomethacin, indoprofen, isofezolac, isoladol, isomethadone, isonixin, isoxepac, isoxicam, ketobemidone, ketoprofen, ketorolac, p-lactophenetide, lefetamine, levorphanol, lofentanil, lonazolac, lomoxicam, loxoprofen, lysine acetylsalicylate, magnesium acetylsalicylate, meclofenamic acid, mefenamic acid, meperidine, meptazinol, mesalamine, metazocine, methadone hydrochloride, methotrimeprazine, metiazinic acid, metofoline, metopon, modafinil, mofebutazone, mofezolac, morazone, morphine, morphine hydrochloride, morphine sulfate, morpholine salicylate, myrophine, nabumetone, nalbuphine, 1-naphthyl salicylate, naproxen, narceine, nefopam, nicomorphine, nifenazone, niflumic acid, nimesulide, 5′-nitro-2′-propoxyacetanilide, norlevorphanol, normethadone, normorphine, norpipanone, olsalazine, opium, oxaceprol, oxametacine, oxaprozin, oxycodone, oxymorphone, oxyphenbutazone, papaveretum, paranyline, parsahnide, pentazocine, perisoxal, phenacetin, phenadoxone, phenazocine, phenazopyridine hydrochloride, phenocoll, phenoperidine, phenopyrazone, phenyl acetylsalicylate, phenylbutazone, phenyl salicylate, phenyramidol, piketoprofen, piminodine, pipebuzone, piperylone, piprofen, pirazolac, piritramide, piroxicam, pranoprofen, proglumetacin, proheptazine, promedol, propacetamol, propiram, propoxyphene, propyphenazone, proquazone, protizinic acid, ramifenazone, remifentanil, rimazolium metilsulfate, salacetamide, salicin, salicylamide, salicylamide o-acetic acid, salicylsulfuric acid, salsalte, salverine, simetride, sodium salicylate, sufentanil, sulfasalazine, sulindac, superoxide dismutase, suprofen, suxibuzone, talniflumate, tenidap, tenoxicam, terofenamate, tetrandrine, thiazolinobutazone, tiaprofenic acid, tiaramide, tilidine, tinoridine, tolfenamic acid, tolmetin, topiramate, tramadol, tropesin, viminol, xenbucin, ximoprofen, zaltoprofen and zomepirac (see The Merck Index, 12th Edition, Therapeutic Category and Biological Activity Index, ed. S. Budavari (1996), pp. Ther-2 to Ther-3 and Ther-12 Analgesic (Dental), Analgesic (Narcotic), Analgesic (Non-narcotic), Anti-inflammatory (Non-steroidal)).

Pharmaceutical compositions of the present invention are useful for treating and preventing inflammation-related cardiovascular disorders, including vascular diseases, coronary artery disease, aneurysm, vascular rejection, arteriosclerosis, atherosclerosis including cardiac transplant atherosclerosis, myocardial infarction, embolism, stroke, thrombosis including venous thrombosis, angina including unstable angina, coronary plaque inflammation, bacterial-induced inflammation including Chlamydia-induced inflammation, viral induced inflammation, and inflammation associated with surgical procedures such as vascular grafting including coronary artery bypass surgery, revascularization procedures including angioplasty, stent placement, endarterectomy, or other invasive procedures involving arteries, veins and capillaries.

These pharmaceutical compositions are also useful in treatment of angiogenesis-related disorders in a subject, for example to inhibit tumor angiogenesis. Such pharmaceutical compositions are useful in treatment of neoplasia, including metastasis; ophthalmological conditions such as corneal graft rejection, ocular neovascularization, retinal neovascularization including neovascularization following injury or infection, diabetic retinopathy, macular degeneration, retrolental fibroplasia and neovascular glaucoma; ulcerative diseases such as gastric ulcer; pathological, but non-malignant, conditions such as hemangiomas, including infantile hemaginomas, angiofibroma of the nasopharynx and avascular necrosis of bone; and disorders of the female reproductive system such as endometriosis.

Moreover, pharmaceutical compositions of the present invention are useful in prevention and treatment of benign and malignant tumors and neoplasia including cancer, such as colorectal cancer, brain cancer, bone cancer, epithelial cell-derived neoplasia (epithelial carcinoma) such as basal cell carcinoma, adenocarcinoma, gastrointestinal cancer such as lip cancer, mouth cancer, esophageal cancer, small bowel cancer, stomach cancer, colon cancer, liver cancer, bladder cancer, pancreatic cancer, ovarian cancer, cervical cancer, lung cancer, breast cancer, skin cancer such as squamous cell and basal cell cancers, prostate cancer, renal cell carcinoma, and other known cancers that effect epithelial cells throughout the body. Neoplasias for which compositions of the invention are contemplated to be particularly useful are gastrointestinal cancer, Barrett's esophagus, liver cancer, bladder cancer, pancreatic cancer, ovarian cancer, prostate cancer, cervical cancer, lung cancer, breast cancer and skin cancer. Such pharmaceutical compositions can also be used to treat fibrosis that occurs with radiation therapy. These pharmaceutical compositions can be used to treat subjects having adenomatous polyps, including those with familial adenomatous polyposis (FAP). Additionally, pharmaceutical compositions of the present invention can be used to prevent polyps from forming in subjects at risk of FAP.

Also, the pharmaceutical compositions inhibit prostanoid-induced smooth muscle contraction by inhibiting synthesis of contractile prostanoids and hence can be of use in treatment of dysmenorrhea, premature labor, asthma and eosinophil-related disorders. They also can be of use for decreasing bone loss particularly in postmenopausal women (i.e., treatment of osteoporosis), and for treatment of glaucoma.

Preferred uses for pharmaceutical compositions of the invention are for treatment of rheumatoid arthritis and osteoarthritis, for pain management generally (particularly post-oral surgery pain, post-general surgery pain, post-orthopedic surgery pain, and acute flares of osteoarthritis), for treatment of Alzheimer's disease, and for colon cancer chemoprevention. A particular preferred use is for rapid pain management, such as when a pharmaceutical composition of the present invention is effective in treating pain within about 30 minutes or less.

Besides being useful for human treatment, pharmaceutical compositions of the invention are useful for veterinary treatment of companion animals, exotic animals, farm animals, and the like, particularly mammals. More particularly, pharmaceutical compositions of the invention are useful for treatment of COX-2 mediated disorders in horses, dogs and cats.

### EXEMPLIFICATION

### Example 1

### Initial Combinatorial Screen

The SFinX™ process was used to find the liquid formulations, which have a solubility of the drug, celecoxib, of at least 200 mg/mL. This general SFinX™ process is outlined in U.S. application Ser. No. 10/700,773, and is herein incorporated in its entirety by reference. The initial step in the SFinX™ process is to perform a single excipient solubility screen to determine which excipients have the ability to solubilize the drug. In this single excipient screen, 77 excipients were tested at four conditions. The conditions were 100 mg/mL at 40 degrees C., 50 mg/mL at 40 degrees C., 50 mg/mL at 60 degrees C. and 50 mg/mL at room temperature. Each excipient was tested in duplicate at each condition. The solubility results from this single excipient screen are provided in Table 1. (Note: The term “SOLID” is used to denote that the excipient is a solid at the specified temperature in Tables 1 and 3. The term “Borderline” is used to denote a cloudy solution of celecoxib in liquid excipients.)

From the list of excipients in Table 1, eight excipients were chosen for a combinatorial ternary excipient solubility screen. These eight excipients were chosen based upon their ability to solubilize celecoxib and also to include a group of chemically diverse excipients. The excipients included were Lauroglycol FCC, poloxamer 331, PEG 400, polyoxyl 20 stearate, polyoxyl 35 castor oil, polysorbate 80, sesame oil, and sorbitan monolaurate. A full combination of the excipients was prepared at a ratio of 64.5: 25.8:9.7. This mixing also resulted in single excipient combinations and binary excipient combinations at the ratios of 90.3:9.7, 64.5:35.5, and 74.2:25.8. This mixing resulted in 8 single excipient combinations, 168 binary combinations, and 336 ternary combinations.

In addition to the ternary mixture screen, eight additional excipients were selected to make binary combinations along with the eight excipients used in the ternary screen. These excipients were Capmul MCM, coconut oil, Softigen 701, triacetin, triethanolamine, vitamin E TPGS, Croval A-70, and olepalisosteariques. These binary excipient combinations were created at a ratio of 3:1. Single excipient combinations also resulted from this screen. This mixing resulted in 16 single excipient combinations and 240 binary combinations.

All excipient mixtures prepared (binary and ternary) were visually screened for their miscibility. From these combinations 314 were determined to be miscible. These miscible combinations were then used to test the solubility of celecoxib.

The solubility of celecoxib was tested at 200 mg/mL at room temperature in the screen. Each formulation was prepared in duplicate. There were 163 formulations that were identified as soluble at 200 mg/ml. There were also an additional 12 formulations that were nearly soluble at 200 mg/ml. These formulation compositions and their respective screen solubility are presented in Table 2.

### Example 2

### Non-Glycol Ether Solubility Screen

An additional combinatorial solubility screen was designed to come up with alternative formulations without the presence of glycol ethers. These excipients and their solubility conditions are listed in Table 3. Seven excipients were screened including: acetylated monoglycerides, monoolein:propylene glycol (90:10), mono-/diglyceride from coconut oil (C8/C10), lecithin, triacetin, triethanolamine, and glyceryl trilaurate. The ratios used to combinatorialize the excipients were 64.5:25.8:9.7, 90.3:9.7, 64.5:36.5, 74.2:25.8, and single excipient combinations. Excipient combinations containing triethanolamine were combinatorialized at the following ratio, 69.6:25.3:5.1, with the last component being triethanolamine. There were 252 excipient combinations that were created by this screen. Of these 252, 94 were miscible after overnight evaluation. One formulation was found to have borderline solubility at 200 mg/mL. In addition, there were four combinations that had definitive solubility at 100 mg/mL and two formulations with a borderline soluble at 100 mg/mL. These formulations are listed in Table 4.

TABLE 1
Single Excipient Solubility Screen of Celecoxib

| | | 100 mg/mL | 50 mg/mL | 50 mg/mL | 50 mg/mL |
|---|---|---|---|---|---|
| | | 40 C. | 40 C. | 60 C. | RT |
| Excipient | Tradename (vendor) | Soluble | Soluble | Soluble | Soluble |
| acetylated | distilled acetylated | NO | NO | YES | SOLID |
| monoglycerides | monoglyceride (Eastman) | | | | |
| monoolein:propylene | Arlacel 186 (Uniqema) | NO | NO | YES | YES |
| glycol (90:10) | | | | | |
| mono-/diglyceride | Capmul MCM (ABITEC) | NO | YES | YES | YES |
| from coconut oil | | | | | |
| (C8/C10) | | | | | |
| propylene glycol | Capryol 90 (Gattefosse) | NO | YES | NO | Borderline |
| monocaprylate | | | | | |
| caprylic/capric | Captex 355 (ABITEC) | NO | NO | NO | NO |
| triglyceride | | | | | |
| C8/C10 diesters of | Captex 200 (ABITEC) | NO | NO | NO | NO |
| propylene glycol of | | | | | |
| coconut oil | | | | | |
| Castor oil | (Sigma) | NO | NO | NO | NO |
| Coconut oil | (Sigma) | NO | NO | NO | NO |
| corn oil | (Sigma) | NO | NO | NO | NO |
| Cottonseed oil | (Sigma) | NO | NO | NO | NO |
| PEG 60 almond | Croval A-70 (Croda) | YES | YES | YES | YES |
| glycerides | | | | | |
| diacetylated | Myvacet 9-45 (Quest) | NO | NO | Borderline | NO |
| monoglycerides | | | | | |
| ethylene glycol | (Aldrich) | NO | NO | NO | NO |
| gelucire 33/01 | (Gattefosse) | NO | NO | NO | NO |
| Glycerin | (Sigma) | NO | NO | NO | NO |
| Glyceryl linoleate | Maisine 35-1 (Gattefosse) | NO | NO | NO | NO |
| glyceryl oleate | Peceol (Gattefosse) | NO | NO | NO | NO |
| Glyceryl ricinoleate | Softigen 701 (Sasol) | NO | NO | YES | YES |
| Hydrogenated coconut | Pureco 100 (ABITEC) | NO | NO | NO | NO |
| oil | | | | | |
| oleoyl macrogol-6 | Labrafil M 1944 CS | NO | Borderline | YES | YES |
| glycerides; apricot | (Gattefosse) | | | | |
| kernel oil peg-6 ester | | | | | |
| linoleoyl macrogol-6 | Labrafil M 2125 CS | NO | Borderline | YES | YES |
| glycerides; corn oil | (Gattefosse) | | | | |
| PEG-6 esters | | | | | |
| PEG-8 caprylic/capric | Labrasol (Gattefosse) | YES | YES | YES | YES |
| glyceride; | | | | | |
| caprylocaproyl | | | | | |
| macrogol-8 glycerides | | | | | |
| Propylene glycol | Lauroglycol FCC | NO | NO | NO | NO |
| monolaurate | (Gattefosse) | | | | |
| Lecithin (high HLB) | Centromix E (Central Soya) | NO | Borderline | YES | YES |
| Lecithin (low HLB) | Centrophase 152 (Central | NO | NO | NO | NO |
| | Soya) | | | | |
| Linoleic acid | (Spectrum) | NO | NO | NO | NO |
| mineral oil | (Aldrich) | NO | NO | NO | NO |
| myristyl alcohol | (Sigma) | SOLID | SOLID | SOLID | SOLID |
| oleic acid | (Spectrum, NF grade) | NO | NO | NO | NO |
| PEG-6 isostearate | Olepal isosteariques | YES | YES | YES | YES |
| | (Gattefosse) | | | | |
| olive oil | (Spectrum) | NO | NO | NO | NO |
| palm oil (palm butter) | (Spectrum) | NO | NO | NO | NO |
| peanut oil | (Sigma) | NO | NO | NO | NO |
| polyglycerol-3- | Plurol diisostearique | NO | NO | NO | NO |
| diisostearate | (Gattefosse) | | | | |
| polyglyceryl-6 dioleate | Plurol Oleique | NO | NO | Borderline | Borderline |
| | CC497(Gattefosse) | | | | |
| POE 26 glycerin | Ethosperse G-26 (Lonza) | YES | YES | YES | YES |
| poloxamer 331 | (Spectrum) | YES | YES | YES | YES |
| polyethylene glycol | (Sigma) | SOLID | SOLID | YES | SOLID |
| 1000 (PEG-20) | | | | | |
| polyethylene glycol | (Sigma) | YES | YES | YES | YES |
| 200 | | | | | |
| polyethylene glycol | (Sigma) | YES | YES | YES | YES |
| 300 | | | | | |
| polyethylene glycol | (Sigma) | YES | YES | YES | YES |
| 400 | | | | | |
| polyethylene glycol | (Sigma) | YES | YES | YES | YES |
| 600 | | | | | |
| Polyoxyl 20 stearate | Myrj 49 PE-NENA | NO | NO | YES | NO |
| | (Uniqema) | | | | |
| Polyoxyl 30 castor oil | Alkamuls EL 620 (Rhodia) | YES | YES | YES | YES |
| Polyoxyl 35 castor oil | Cremophor EL (BASF) | YES | YES | YES | YES |
| Polyoxyl 40 castor oil | Alkamuls EL 719 (Rhodia) | YES | YES | YES | YES |
| Polyoxyl 40 | Cremophor RH40 (BASF) | YES | YES | YES | NO |
| Hydrogenated castor | | | | | |
| oil | | | | | |
| Polyoxyl 40 stearate | Myrj 52 (Sigma) | SOLID | SOLID | YES | SOLID |
| Polypropylene glycol | (Aldrich) | YES | YES | YES | YES |
| (MW 725) | | | | | |
| Polypropylene glycol | (Aldrich) | YES | YES | YES | YES |
| (MW 2000) | | | | | |
| Polysorbate 20 | (Sigma) | YES | YES | YES | YES |
| Polysorbate 40 | (Spectrum) | YES | YES | YES | YES |
| Polysorbate 80 | (Sigma) | YES | YES | YES | YES |
| Polysorbate60 | Tween 60 (Aldrich) | YES | YES | YES | YES |
| Propylene glycol | (Sigma) - USP grade | NO | Borderline | YES | YES |
| Safflower oil | (Spectrum) | NO | NO | NO | NO |
| Sesame oil | (Sigma) | NO | NO | NO | NO |
| Sorbitan monolaurate | Span 20 (Sigma) | NO | NO | YES | YES |
| Sorbitan monooleate | Span 80 (Sigma) | NO | NO | Borderline | Borderline |
| Sorbitan trioleate | Span 85 (Spectrum) | NO | NO | NO | NO |
| Soybean oil | (Sigma) | NO | NO | NO | NO |
| Sunflower seed oil | (Sigma) | NO | NO | NO | NO |
| polyoxyethylene | Tagat TO (Goldschmidt) | YES | YES | YES | YES |
| glycerol trioleate | | | | | |
| Tocopherol | Sigma | NO | NO | NO | NO |
| Triacetin | (Aldrich) | YES | YES | YES | YES |
| Triethanolamine | Spectrum | YES | YES | YES | YES |
| (Trolamine) | | | | | |
| Trilaurin (glyceryl | (Lipo) | SOLID | SOLID | YES | SOLID |
| trilaurate) | | | | | |
| Vegetable oil (partially | BBS-C (ABITEC) | SOLID | SOLID | NO | SOLID |
| hydrogenated & | | | | | |
| hydrogenated) | | | | | |
| Vitamin E TPGS | Vitamin E TPGS (Eastman) | SOLID | SOLID | YES | SOLID |
| (Eastman) | | | | | |
| benzyl alcohol | benzyl alcohol (Sigma) | YES | YES | Borderline | NO |
| benzyl benzoate | (Sigma) | Borderline | YES | Borderline | NO |
| Isopropanolamine(1- | (Aldrich) | YES | YES | YES | YES |
| amino-2-propanol) | | | | | |

TABLE 2
Combinatorial Solubility Screen of Celecoxib at Room Temperature

| | | | | | | Soluble at 200 |
|---|---|---|---|---|---|---|
| Excipient 1 | % | Excipient 2 | % | Excipient 3 | % | mg/mL |
| Lauroglycol FCC | 64.5 | PEG 400 | 25.8 | Lauroglycol FCC | 9.7 | YES |
| Lauroglycol FCC | 64.5 | PEG 400 | 25.8 | Polysorbate 80 | 9.7 | YES |
| Lauroglycol FCC | 64.5 | PEG 400 | 25.8 | Span 20 | 9.7 | YES |
| Lauroglycol FCC | 64.5 | Polyoxyl 35 Castor Oil | 25.8 | Poloxamer 331 | 9.7 | YES |
| Lauroglycol FCC | 64.5 | Polyoxyl 35 Castor Oil | 25.8 | PEG 400 | 9.7 | YES |
| Lauroglycol FCC | 64.5 | Polyoxyl 35 Castor Oil | 25.8 | PEG 20 Stearate | 9.7 | YES |
| Lauroglycol FCC | 64.5 | Polyoxyl 35 Castor Oil | 25.8 | Polysorbate 80 | 9.7 | Borderline |
| Lauroglycol FCC | 64.5 | Polysorbate 80 | 25.8 | PEG 400 | 9.7 | YES |
| Lauroglycol FCC | 64.5 | Polysorbate 80 | 25.8 | PEG 20 Stearate | 9.7 | YES |
| Lauroglycol FCC | 64.5 | Polysorbate 80 | 25.8 | Polyoxyl 35 Castor Oil | 9.7 | YES |
| Lauroglycol FCC | 64.5 | Polysorbate 80 | 25.8 | Polysorbate 80 | 9.7 | Borderline |
| Poloxamer 331 | 64.5 | Lauroglycol FCC | 25.8 | Lauroglycol FCC | 9.7 | Borderline |
| Poloxamer 331 | 64.5 | Lauroglycol FCC | 25.8 | Poloxamer 331 | 9.7 | YES |
| Poloxamer 331 | 64.5 | Lauroglycol FCC | 25.8 | Polysorbate 80 | 9.7 | YES |
| Poloxamer 331 | 64.5 | Poloxamer 331 | 25.8 | Lauroglycol FCC | 9.7 | YES |
| Poloxamer 331 | 64.5 | Poloxamer 331 | 25.8 | Poloxamer 331 | 9.7 | YES |
| Poloxamer 331 | 64.5 | Poloxamer 331 | 25.8 | Sesame Oil | 9.7 | YES |
| Poloxamer 331 | 64.5 | Polysorbate 80 | 25.8 | Span 20 | 9.7 | YES |
| Poloxamer 331 | 64.5 | Sesame Oil | 25.8 | Lauroglycol FCC | 9.7 | YES |
| Poloxamer 331 | 64.5 | Sesame Oil | 25.8 | Poloxamer 331 | 9.7 | YES |
| PEG 400 | 64.5 | PEG 400 | 25.8 | PEG 400 | 9.7 | YES |
| PEG 400 | 64.5 | PEG 400 | 25.8 | Span 20 | 9.7 | YES |
| PEG 400 | 64.5 | Polyoxyl 35 Castor Oil | 25.8 | Lauroglycol FCC | 9.7 | YES |
| PEG 400 | 64.5 | Span 20 | 25.8 | Span 20 | 9.7 | YES |
| Span 20 | 64.5 | PEG 400 | 25.8 | Lauroglycol FCC | 9.7 | YES |
| Span 20 | 64.5 | PEG 400 | 25.8 | Poloxamer 331 | 9.7 | YES |
| Span 20 | 64.5 | PEG 400 | 25.8 | PEG 400 | 9.7 | YES |
| Span 20 | 64.5 | PEG 400 | 25.8 | Polyoxyl 35 Castor Oil | 9.7 | YES |
| Span 20 | 64.5 | PEG 400 | 25.8 | Polysorbate 80 | 9.7 | YES |
| Span 20 | 64.5 | PEG 400 | 25.8 | Sesame Oil | 9.7 | Borderline |
| Span 20 | 64.5 | PEG 400 | 25.8 | Span 20 | 9.7 | Borderline |
| Span 20 | 64.5 | Polysorbate 80 | 25.8 | PEG 400 | 9.7 | YES |
| Polyoxyl 35 Castor Oil | 64.5 | Lauroglycol FCC | 25.8 | Lauroglycol FCC | 9.7 | YES |
| Polyoxyl 35 Castor Oil | 64.5 | Lauroglycol FCC | 25.8 | Poloxamer 331 | 9.7 | YES |
| Polyoxyl 35 Castor Oil | 64.5 | Lauroglycol FCC | 25.8 | PEG 400 | 9.7 | YES |
| Polyoxyl 35 Castor Oil | 64.5 | Lauroglycol FCC | 25.8 | Polyoxyl 35 Castor Oil | 9.7 | YES |
| Polyoxyl 35 Castor Oil | 64.5 | Lauroglycol FCC | 25.8 | Polysorbate 80 | 9.7 | YES |
| Polyoxyl 35 Castor Oil | 64.5 | Lauroglycol FCC | 25.8 | Sesame Oil | 9.7 | YES |
| Polyoxyl 35 Castor Oil | 64.5 | Lauroglycol FCC | 25.8 | Span 20 | 9.7 | YES |
| Polyoxyl 35 Castor Oil | 64.5 | Poloxamer 331 | 25.8 | PEG 400 | 9.7 | YES |
| Polyoxyl 35 Castor Oil | 64.5 | Poloxamer 331 | 25.8 | Polyoxyl 35 Castor Oil | 9.7 | YES |
| Polyoxyl 35 Castor Oil | 64.5 | Poloxamer 331 | 25.8 | Polysorbate 80 | 9.7 | YES |
| Polyoxyl 35 Castor Oil | 64.5 | Poloxamer 331 | 25.8 | Sesame Oil | 9.7 | YES |
| Polyoxyl 35 Castor Oil | 64.5 | Poloxamer 331 | 25.8 | Span 20 | 9.7 | YES |
| Polyoxyl 35 Castor Oil | 64.5 | PEG 400 | 25.8 | Lauroglycol FCC | 9.7 | YES |
| Polyoxyl 35 Castor Oil | 64.5 | PEG 400 | 25.8 | Poloxamer 331 | 9.7 | YES |
| Polyoxyl 35 Castor Oil | 64.5 | PEG 400 | 25.8 | PEG 400 | 9.7 | YES |
| Polyoxyl 35 Castor Oil | 64.5 | PEG 400 | 25.8 | Polyoxyl 35 Castor Oil | 9.7 | YES |
| Polyoxyl 35 Castor Oil | 64.5 | PEG 400 | 25.8 | Polysorbate 80 | 9.7 | YES |
| Polyoxyl 35 Castor Oil | 64.5 | PEG 400 | 25.8 | Sesame Oil | 9.7 | YES |
| Polyoxyl 35 Castor Oil | 64.5 | PEG 400 | 25.8 | Span 20 | 9.7 | YES |
| Polyoxyl 35 Castor Oil | 64.5 | Polyoxyl 35 Castor Oil | 25.8 | Lauroglycol FCC | 9.7 | YES |
| Polyoxyl 35 Castor Oil | 64.5 | Polyoxyl 35 Castor Oil | 25.8 | Poloxamer 331 | 9.7 | YES |
| Polyoxyl 35 Castor Oil | 64.5 | Polyoxyl 35 Castor Oil | 25.8 | PEG 400 | 9.7 | YES |
| Polyoxyl 35 Castor Oil | 64.5 | Polyoxyl 35 Castor Oil | 25.8 | Polyoxyl 35 Castor Oil | 9.7 | YES |
| Polyoxyl 35 Castor Oil | 64.5 | Polyoxyl 35 Castor Oil | 25.8 | Polysorbate 80 | 9.7 | YES |
| Polyoxyl 35 Castor Oil | 64.5 | Polyoxyl 35 Castor Oil | 25.8 | Sesame Oil | 9.7 | YES |
| Polyoxyl 35 Castor Oil | 64.5 | Polyoxyl 35 Castor Oil | 25.8 | Span 20 | 9.7 | YES |
| Polyoxyl 35 Castor Oil | 64.5 | Polysorbate 80 | 25.8 | Lauroglycol FCC | 9.7 | YES |
| Polyoxyl 35 Castor Oil | 64.5 | Polysorbate 80 | 25.8 | Poloxamer 331 | 9.7 | YES |
| Polyoxyl 35 Castor Oil | 64.5 | Polysorbate 80 | 25.8 | PEG 400 | 9.7 | YES |
| Polyoxyl 35 Castor Oil | 64.5 | Polysorbate 80 | 25.8 | Polyoxyl 35 Castor Oil | 9.7 | YES |
| Polyoxyl 35 Castor Oil | 64.5 | Polysorbate 80 | 25.8 | Polysorbate 80 | 9.7 | YES |
| Polyoxyl 35 Castor Oil | 64.5 | Polysorbate 80 | 25.8 | Sesame Oil | 9.7 | YES |
| Polyoxyl 35 Castor Oil | 64.5 | Polysorbate 80 | 25.8 | Span 20 | 9.7 | YES |
| Polyoxyl 35 Castor Oil | 64.5 | Span 20 | 25.8 | Lauroglycol FCC | 9.7 | YES |
| Polyoxyl 35 Castor Oil | 64.5 | Span 20 | 25.8 | Poloxamer 331 | 9.7 | YES |
| Polyoxyl 35 Castor Oil | 64.5 | Span 20 | 25.8 | PEG 400 | 9.7 | YES |
| Polyoxyl 35 Castor Oil | 64.5 | Span 20 | 25.8 | Polyoxyl 35 Castor Oil | 9.7 | YES |
| Polyoxyl 35 Castor Oil | 64.5 | Span 20 | 25.8 | Polysorbate 80 | 9.7 | YES |
| Polyoxyl 35 Castor Oil | 64.5 | Span 20 | 25.8 | Sesame Oil | 9.7 | YES |
| Polyoxyl 35 Castor Oil | 64.5 | Span 20 | 25.8 | Span 20 | 9.7 | YES |
| Polysorbate 80 | 64.5 | Lauroglycol FCC | 25.8 | Lauroglycol FCC | 9.7 | YES |
| Polysorbate 80 | 64.5 | Lauroglycol FCC | 25.8 | PEG 400 | 9.7 | YES |
| Polysorbate 80 | 64.5 | Lauroglycol FCC | 25.8 | Polyoxyl 35 Castor Oil | 9.7 | YES |
| Polysorbate 80 | 64.5 | Lauroglycol FCC | 25.8 | Polysorbate 80 | 9.7 | YES |
| Polysorbate 80 | 64.5 | Lauroglycol FCC | 25.8 | Sesame Oil | 9.7 | YES |
| Polysorbate 80 | 64.5 | Lauroglycol FCC | 25.8 | Span 20 | 9.7 | YES |
| Polysorbate 80 | 64.5 | Poloxamer 331 | 25.8 | PEG 400 | 9.7 | YES |
| Polysorbate 80 | 64.5 | Poloxamer 331 | 25.8 | Polysorbate 80 | 9.7 | YES |
| Polysorbate 80 | 64.5 | PEG 400 | 25.8 | Lauroglycol FCC | 9.7 | YES |
| Polysorbate 80 | 64.5 | PEG 400 | 25.8 | Poloxamer 331 | 9.7 | YES |
| Polysorbate 80 | 64.5 | PEG 400 | 25.8 | PEG 400 | 9.7 | YES |
| Polysorbate 80 | 64.5 | PEG 400 | 25.8 | Polyoxyl 35 Castor Oil | 9.7 | YES |
| Polysorbate 80 | 64.5 | PEG 400 | 25.8 | Polysorbate 80 | 9.7 | YES |
| Polysorbate 80 | 64.5 | PEG 400 | 25.8 | Sesame Oil | 9.7 | YES |
| Polysorbate 80 | 64.5 | PEG 400 | 25.8 | Span 20 | 9.7 | YES |
| Polysorbate 80 | 64.5 | Polyoxyl 35 Castor Oil | 25.8 | Lauroglycol FCC | 9.7 | YES |
| Polysorbate 80 | 64.5 | Polyoxyl 35 Castor Oil | 25.8 | Poloxamer 331 | 9.7 | YES |
| Polysorbate 80 | 64.5 | Polyoxyl 35 Castor Oil | 25.8 | PEG 400 | 9.7 | YES |
| Polysorbate 80 | 64.5 | Polyoxyl 35 Castor Oil | 25.8 | Polyoxyl 35 Castor Oil | 9.7 | YES |
| Polysorbate 80 | 64.5 | Polyoxyl 35 Castor Oil | 25.8 | Polysorbate 80 | 9.7 | YES |
| Polysorbate 80 | 64.5 | Polyoxyl 35 Castor Oil | 25.8 | Sesame Oil | 9.7 | YES |
| Polysorbate 80 | 64.5 | Polyoxyl 35 Castor Oil | 25.8 | Span 20 | 9.7 | YES |
| Polysorbate 80 | 64.5 | Polysorbate 80 | 25.8 | Lauroglycol FCC | 9.7 | YES |
| Polysorbate 80 | 64.5 | Polysorbate 80 | 25.8 | Poloxamer 331 | 9.7 | YES |
| Polysorbate 80 | 64.5 | Polysorbate 80 | 25.8 | PEG 400 | 9.7 | YES |
| Polysorbate 80 | 64.5 | Polysorbate 80 | 25.8 | Polyoxyl 35 Castor Oil | 9.7 | YES |
| Polysorbate 80 | 64.5 | Polysorbate 80 | 25.8 | Polysorbate 80 | 9.7 | YES |
| Polysorbate 80 | 64.5 | Polysorbate 80 | 25.8 | Sesame Oil | 9.7 | YES |
| Polysorbate 80 | 64.5 | Polysorbate 80 | 25.8 | Span 20 | 9.7 | YES |
| Polysorbate 80 | 64.5 | Span 20 | 25.8 | Lauroglycol FCC | 9.7 | YES |
| Polysorbate 80 | 64.5 | Span 20 | 25.8 | Poloxamer 331 | 9.7 | YES |
| Polysorbate 80 | 64.5 | Span 20 | 25.8 | PEG 400 | 9.7 | YES |
| Polysorbate 80 | 64.5 | Span 20 | 25.8 | Polyoxyl 35 Castor Oil | 9.7 | YES |
| Polysorbate 80 | 64.5 | Span 20 | 25.8 | Polysorbate 80 | 9.7 | YES |
| Polysorbate 80 | 64.5 | Span 20 | 25.8 | Sesame Oil | 9.7 | YES |
| Polysorbate 80 | 64.5 | Span 20 | 25.8 | Span 20 | 9.7 | YES |
| Polyoxyl 35 Castor Oil | 75 | Lauroglycol FCC | 25 | | | YES |
| Polysorbate 80 | 75 | Lauroglycol FCC | 25 | | | YES |
| Poloxamer 331 | 75 | Poloxamer 331 | 25 | | | YES |
| Polysorbate 80 | 75 | Poloxamer 331 | 25 | | | YES |
| PEG 400 | 75 | PEG 400 | 25 | | | YES |
| Polyoxyl 35 Castor Oil | 75 | PEG 400 | 25 | | | YES |
| Polysorbate 80 | 75 | PEG 400 | 25 | | | YES |
| Polyoxyl 35 Castor Oil | 75 | Polyoxyl 35 Castor Oil | 25 | | | YES |
| Polysorbate 80 | 75 | Polyoxyl 35 Castor Oil | 25 | | | YES |
| PEG 400 | 75 | Polysorbate 80 | 25 | | | YES |
| Polyoxyl 35 Castor Oil | 75 | Polysorbate 80 | 25 | | | YES |
| Polysorbate 80 | 75 | Polysorbate 80 | 25 | | | YES |
| Poloxamer 331 | 75 | Sesame Oil | 25 | | | Borderline |
| PEG 400 | 75 | Span 20 | 25 | | | YES |
| Polyoxyl 35 Castor Oil | 75 | Span 20 | 25 | | | YES |
| Polysorbate 80 | 75 | Span 20 | 25 | | | YES |
| Poloxamer 331 | 75 | Capmul MCM | 25 | | | Borderline |
| PEG 400 | 75 | Capmul MCM | 25 | | | YES |
| Polyoxyl 35 Castor Oil | 75 | Capmul MCM | 25 | | | YES |
| Polysorbate 80 | 75 | Capmul MCM | 25 | | | YES |
| PEG 400 | 75 | Softigen 701 | 25 | | | YES |
| Polyoxyl 35 Castor Oil | 75 | Softigen 701 | 25 | | | YES |
| Polysorbate 80 | 75 | Softigen 701 | 25 | | | YES |
| Poloxamer 331 | 75 | Triacetin | 25 | | | YES |
| PEG 400 | 75 | Triacetin | 25 | | | YES |
| Polyoxyl 35 Castor Oil | 75 | Triacetin | 25 | | | YES |
| Polysorbate 80 | 75 | Triacetin | 25 | | | YES |
| PEG 400 | 75 | Triethanolamine | 25 | | | YES |
| Polyoxyl 35 Castor Oil | 75 | Tocopherol | 25 | | | YES |
| Polysorbate 80 | 75 | Tocopherol | 25 | | | YES |
| PEG 400 | 75 | Croval A-70 | 25 | | | YES |
| Polyoxyl 35 Castor Oil | 75 | Croval A-70 | 25 | | | YES |
| Polysorbate 80 | 75 | Croval A-70 | 25 | | | YES |
| Poloxamer 331 | 75 | Olepal isosteariques | 25 | | | YES |
| Polyoxyl 35 Castor Oil | 75 | Olepal isosteariques | 25 | | | YES |
| Croval A-70 | 75 | Lauroglycol FCC | 25 | | | YES |
| Olepal isosteariques | 75 | Lauroglycol FCC | 25 | | | YES |
| Olepal isosteariques | 75 | Poloxamer 331 | 25 | | | YES |
| Capmul MCM | 75 | PEG 400 | 25 | | | YES |
| Triacetin | 75 | PEG 400 | 25 | | | YES |
| Triethanolamine | 75 | PEG 400 | 25 | | | YES |
| Tocopherol | 75 | PEG 400 | 25 | | | YES |
| Croval A-70 | 75 | PEG 400 | 25 | | | YES |
| Capmul MCM | 75 | PEG 20 Stearate | 25 | | | Borderline |
| Triacetin | 75 | Polyoxyl 35 Castor Oil | 25 | | | YES |
| Croval A-70 | 75 | Polyoxyl 35 Castor Oil | 25 | | | YES |
| Olepal isosteariques | 75 | Polyoxyl 35 Castor Oil | 25 | | | YES |
| Softigen 701 | 75 | Polysorbate 80 | 25 | | | Borderline |
| Croval A-70 | 75 | Polysorbate 80 | 25 | | | YES |
| Olepal isosteariques | 75 | Polysorbate 80 | 25 | | | YES |
| Olepal isosteariques | 75 | Sesame Oil | 25 | | | Borderline |
| Croval A-70 | 75 | Span 20 | 25 | | | YES |
| Triethanolamine | 75 | Capmul MCM | 25 | | | Borderline |
| Croval A-70 | 75 | Capmul MCM | 25 | | | YES |
| Olepal isosteariques | 75 | Capmul MCM | 25 | | | YES |
| Olepal isosteariques | 75 | Coconut Oil | 25 | | | YES |
| Croval A-70 | 75 | Softigen 701 | 25 | | | YES |
| Olepal isosteariques | 75 | Softigen 701 | 25 | | | YES |
| Croval A-70 | 75 | Triacetin | 25 | | | YES |
| Olepal isosteariques | 75 | Triacetin | 25 | | | YES |
| Croval A-70 | 75 | Tocopherol | 25 | | | YES |
| Olepal isosteariques | 75 | Tocopherol | 25 | | | Borderline |
| Triacetin | 75 | Croval A-70 | 25 | | | YES |
| Olepal isosteariques | 75 | Croval A-70 | 25 | | | YES |
| Triacetin | 75 | Olepal isosteariques | 25 | | | YES |
| Croval A-70 | 75 | Olepal isosteariques | 25 | | | YES |
| Olepal isosteariques | 75 | Olepal isosteariques | 25 | | | YES |

TABLE 3
Single Excipient Solubility Screen of Celecoxib

| | | 100 mg/mL | 50 mg/mL | 50 mg/mL | 50 mg/mL |
|---|---|---|---|---|---|
| | | 40 C. | 40 C. | 60 C. | RT |
| Excipient | Tradename (vendor) | Soluble | Soluble | Soluble | Soluble |
| acetylated monoglycerides | distilled acetylated | NO | NO | YES | SOLID |
| | monoglyceride (Eastman) | | | | |
| monoolein:propylene glycol (90:10) | Arlacel 186 (Uniqema) | NO | NO | YES | YES |
| mono-/diglyceride from coconut oil | Capmul MCM (ABITEC) | NO | YES | YES | YES |
| (C8/C10) | | | | | |
| Glyceryl ricinoleate | Softigen 701 (Sasol) | NO | NO | YES | YES |
| Lecithin (high HLB) | Centromix E (Central | NO | Borderline | YES | YES |
| | Soya) | | | | |
| polyglyceryl-6 dioleate | Plurol Oleique | NO | NO | Borderline | Borderline |
| | CC497(Gattefosse) | | | | |
| Propylene glycol | | NO | Borderline | YES | YES |
| Triacetin | | YES | YES | YES | YES |
| Triethanolamine | Trolamine (Spectrum) | YES | YES | YES | YES |
| glyceryl trilaurate | Trilaurin (Lipo) | SOLID | SOLID | YES | SOLID |
| Isopropanolamine | | YES | YES | YES | YES |

TABLE 4
Combinatorial Solubility Screen of Celecoxib at Room Temperature

| | | | | | | 400 | 200 | 100 |
|---|---|---|---|---|---|---|---|---|
| Excipient 1 | % | Excipient 2 | % | Excipient 3 | % | mg/mL | mg/mL | mg/mL |
| Capmul MCM | 64.5 | Triacetin | 25.8 | Arlacel 186 | 9.7 | NO | NO | YES |
| Triacetin | 64.5 | acetyleated | 25.8 | acetyleated | 9.7 | NO | NO | Borderline |
| | | monoglycerides | | monoglycerides | | | | |
| Triacetin | 64.5 | acetyleated | 25.8 | Trilaurin | 9.7 | NO | NO | Borderline |
| | | monoglycerides | | | | | | |
| Triacetin | 64.5 | Arlacel 186 | 25.8 | acetyleated | 9.7 | NO | NO | YES |
| | | | | monoglycerides | | | | |
| Triacetin | 64.5 | Triacetin | 25.8 | acetyleated | 9.7 | NO | NO | YES |
| | | | | monoglycerides | | | | |
| Triacetin | 64.5 | Triacetin | 25.8 | Triacetin | 9.7 | NO | NO | YES |
| Triacetin | 69.6 | Triacetin | 25.3 | Triethanolamine | 5.1 | NO | Borderline | YES |

While this invention has been particularly shown and described with references to preferred embodiments thereof, it will be understood by those skilled in the art that various changes in form and details may be made therein without departing from the scope of the invention encompassed by the appended claims.

1. A pharmaceutical composition comprising celecoxib; a first excipient which is a propylene glycol fatty acid monoester; a second excipient selected from the group consisting of a polyethylene glycol, a polyoxyl castor oil, and a polysorbate; and a third excipient selected from the group consisting of a propylene glycol fatty acid monoester, a polysorbate, a sorbitan fatty acid monoester, a poloxamer, a polyethylene glycol, a polyethylene glycol fatty acid monoester, and a polyoxyl castor oil.

2-82. (canceled)

83. The pharmaceutical composition of claim 1, wherein:
(a) said second excipient is selected from the group consisting of polyethylene glycol 400, polyoxyl 35 castor oil and polysorbate 80;
(b) said third excipient is selected from the group consisting of propylene glycol monolaurate, polysorbate 80, sorbitan monolaurate, poloxamer 331, polyethylene glycol 400 and polyethylene glycol 20 stearate;
(c) said first excipient is propylene glycol monolaurate;
(d) said first excipient comprises about 60% to about 70% by weight of the first, second and third excipients; or
(e) said second excipient comprises about 20% to about 30% by weight of the first, second and third excipients and the third excipient comprises about 5% to about 15% by weight of the first, second and third excipients.

84. A pharmaceutical composition comprising celecoxib; a first excipient which is a poloxamer; a second excipient selected from the group consisting of a propylene glycol fatty acid monoester, a polyoxamer, and a plant oil; and a third excipient selected from the group consisting of a propylene glycol fatty acid monoester, a poloxamer, a polysorbate and a sorbitan fatty acid monoester.

85. The pharmaceutical composition of claim 84, wherein:
(a) said second excipient is selected from the group consisting of propylene glycol monolaurate, poloxamer 331 and sesame oil;
(b) said third excipient is selected from the group consisting of propylene glycol monolaurate, poloxamer 331, polysorbate 80, sesame oil and sorbital monolaurate;
(c) said first excipient is poloxamer 331;
(d) said first excipient comprises about 60% to about 70% by weight of the first, second and third excipients; or
(e) said second excipient comprises about 20% to about 30% by weight of the first, second and third excipients and the third excipient comprises about 5% to about 15% by weight of the first, second and third excipients.

86. A pharmaceutical composition comprising celecoxib; a first excipient which is a polyethylene glycol; a second excipient selected from the group consisting of a polyethylene glycol, a polyoxyl castor oil and a sorbitan fatty acid monoester; and a third excipient selected from the group consisting of a polyethylene glycol, a sorbitan fatty acid monoester and a propylene glycol fatty acid monoester.

87. The pharmaceutical composition of claim 86, wherein:
(a) said second excipient is selected from the group consisting of polyethylene glycol 400, polyoxyl 35 castor oil and sorbitan monolaurate;
(b) said third excipient is selected from the group consisting of polyethylene glycol 400, sorbitan monolaurate and propylene glycol monolaurate;
(c) said first excipient is polyethylene glycol 400;
(d) said first excipient comprises about 60% to about 70% by weight of the first, second and third excipients; or
(e) said second excipient comprises about 20% to about 30% by weight of the first, second and third excipients and the third excipient comprises about 5% to about 15% by weight of the first, second and third excipients.

88. A pharmaceutical composition comprising celecoxib; a first excipient which is a sorbitan fatty acid monoester; a second excipient selected from the group consisting of a polyethylene glycol and a polysorbate; and a third excipient selected from the group consisting of a propylene glycol fatty acid monoester, a polyoxamer, a polyethylene glycol, a polyoxyl castor oil, a polysorbate, a plant oil or a sorbitan fatty acid monoester.

89. The pharmaceutical composition of claim 88, wherein:
(a) said second excipient is selected from the group consisting of polyethylene glycol 400 and polysorbate 80;
(b) said third excipient is selected from the group of propylene glycol laurate, polyoxamer 331, polyethylene glycol 400, polyoxyl 35 castor oil, polysorbate 80, sesame oil and sorbitan monolaurate;
(c) said first excipient is sorbitan monolaurate;
(d) said first excipient comprises about 60% to about 70% by weight of the first, second and third excipients; or
(e) said second excipient comprises about 20% to about 30% by weight of the first, second and third excipients and the third excipient comprises about 5% to about 15% by weight of the first, second and third excipients.

90. A pharmaceutical composition comprising celecoxib; a first excipient which is a polyoxyl castor oil; a second excipient selected from the group consisting of a propylene glycol fatty acid monoester, a poloxamer, a polyethylene glycol, a polyoxyl castor oil, a polysorbate and a sorbitan fatty acid monoester; and a third excipient selected from the group consisting of a propylene glycol fatty acid monoester, a polyoxamer, a polyethylene glycol, a polyoxyl castor oil, a polysorbate, a plant oil and a sorbitan fatty acid monoester.

91. The pharmaceutical composition of claim 90, wherein:
(a) said second excipient is selected from the group consisting of propylene glycol monolaurate, poloxamer 331, polyethylene glycol 400, polyoxyl 35 castor oil, polysorbate 80 and sorbitan monolaurate;
(b) said third excipient is selected from the group consisting of propylene glycol monolaurate, poloxamer 331, polyethylene glycol 400, polyoxyl 35 castor oil, polysorbate 80, sorbitan monolaurate and sesame oil;
(c) said first excipient is polyoxyl 35 castor oil;
(d) said first excipient comprises about 60% to about 70% by weight of the first, second and third excipients; or
(e) said second excipient comprises about 20% to about 30% by weight of the first, second and third excipients and the third excipient comprises about 5% to about 15% by weight of the first, second and third excipients.

92. A pharmaceutical composition comprising celecoxib; a first excipient which is a polysorbate; a second excipient selected from the group consisting of a propylene glycol fatty acid monoester, a poloxamer, a polyethylene glycol, a polyoxyl castor oil, a polysorbate and a sorbitan fatty acid monoester; and a third excipient selected from the group consisting of a propylene glycol fatty acid monoester, a polyethylene glycol, a polyoxyl castor oil, a polysorbate a plant oil, a sorbitan fatty acid monoester and a poloxamer.

93. The pharmaceutical composition of claim 92, wherein:
(a) said second excipient is selected from the group consisting of propylene glycol monolaurate, poloxamer 331, polyethylene glycol 400, polyoxyl 35 castor oil, polysorbate 80 and sorbitan monolaurate;
(b) said third excipient is selected from the group consisting of propylene glycol monolaurate, poloxamer 331, polyethylene glycol 400, polyoxyl 35 castor oil, polysorbate 80, sorbitan monolaurate and sesame oil;
(c) said first excipient is polysorbate 80;
(d) said first excipient comprises about 60% to about 70% by weight of the first, second and third excipients; or
(e) said second excipient comprises about 20% to about 30% by weight of the first, second and third excipients and the third excipient comprises about 5% to about 15% by weight of the first, second and third excipients.
